SEQUENCE LISTING (8 sequences)

SEQ ID NO: 1 - DNA, 36 bp, Artificial sequence
  Chemically synthesized nucleic acid sequence of a target for the HMPTBL16 probe of the invention
gctcatcagcaaaatgagggaggagtacccagacag

SEQ ID NO: 2 - DNA, 37 bp, Artificial sequence
  Chemically synthesized nucleic acid sequence of a target for a probe of the invention
gctcatcagcaaaatgagtggaggagtacccagacag

SEQ ID NO: 3 - DNA, 39 bp, Artificial sequence
  Chemically synthesized nucleic acid sequence of a target for a probe of the invention
gctcatcagcaaaatgagtttggaggagtacccagacag

SEQ ID NO: 4 - DNA, 41 bp, Artificial sequence
  Chemically synthesized nucleic acid sequence of a target for a probe of the invention
gctcatcagcaaaatgagtttttggaggagtacccagacag

SEQ ID NO: 5 - DNA, 43 bp, Artificial sequence
  Chemically synthesized nucleic acid sequence of a target for a probe of the invention
gctcatcagcaaaatgagtttttttggaggagtacccagacag

SEQ ID NO: 6 - DNA, 37 bp, Artificial sequence
  Chemically synthesized nucleic acid sequence that was synthesized to target Tublin mRNA(516-551)
ctcattttgctgatgagcnctgtctgggtactcctcc

SEQ ID NO: 7 - DNA, 37 bp, Artificial sequence; identical to SEQ ID NO: 6
  Chemically synthesized nucleic acid sequence of a probe (HMPTBL16 probe)

SEQ ID NO: 8 - DNA, 36 bp, Artificial sequence
  Chemically synthesized nucleic acid sequence of a control for the HMPTBL16 probe
tctgtgtaatcaactgggagaatgtaactgactagc

(19) United States
(12) Patent Application Publication
(10) Pub. No.: US 2009/0156416 A1
(43) Pub. Date: Jun. 18, 2009

(54) Hybrid Molecular Probe

(76) Inventors: Weihong Tan, Gainesville, FL (US); Chaoyong Yang, El Cerrito, CA (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/994,965

(22) PCT Filed: Oct. 11, 2006
(86) PCT No.: PCT/US06/40082

§ 371 Date: Sep. 3, 2008

Related U.S. Application Data
(60) Provisional application No. 60/726,306, filed on Oct. 12, 2005.

(51) Int. Cl. C40B 30/04; C07H 21/00; C07K 14/00; G01N 33/48

(52) U.S. Cl. 506/9; 536/24.3; 530/402; 436/94

(57) ABSTRACT

A system and method for analyzing a substance, in particular RNA in vivo, comprising a hybrid molecular probe, said probe comprising two single-stranded nucleic acid sequences tethered together with a polyethylene glycol polymer and fluorophores attached to either end of the sequences. When a probe of the invention hybridizes to a target substance (such as a target RKA sequence), fluorescence resonance energy transfer occurs between the two fluorophores to generate a visible signal.

### GOVERNMENT SUPPORT

The subject matter of this application has been supported in part by U.S. Government Support under NIH GM66137; NIH NS045174; and NSF EF0304569. Accordingly, the U.S. Government has certain rights in this invention.

### FIELD OF THE INVENTION

The present invention provides a novel molecular probe that has the ability to bind to a target nucleic acid sequence with a high degree of sensitivity and selectivity, whereupon binding of the probe to a target sequence produces a detectable signal for use in real-time monitoring of in vivo activities.

### BACKGROUND OF THE INVENTION

Techniques that allow specific detection of target molecules or oligonucleotides are important for many areas of research, as well as for clinical diagnostics. Central to most detection techniques are ligands that dictate specific and high affinity binding to a target molecule of interest. In immunodiagnostic assays, antibodies mediate specific and high affinity binding, whereas in assays detecting nucleic acid target sequences, complementary oligonucleotide probes fulfill this role. To date, antibodies have been able to provide molecular recognition needs for a wide variety of target molecules and have been the popular choice of the class of ligands for developing diagnostic assays.

Messenger RNA performs an important role in gene expression by carrying genetic information from DNA to protein synthesis machinery. Ways to monitor the synthesis, transportation, localization of mRNA in living cells especially in real-time will offer insight understanding in molecular biology, facilitate drug discovery, and revolutionize disease diagnostics and treatments. Traditional RNA analysis methods such as in situ hybridization lack the ability to monitor dynamic cellular events such as synthesis and transportation of mRNA. To offer real-time monitoring capability, molecular probes have to be developed that are able to tansduce target recognition directly into a signal distinguishable from background with high sensitivity and selectivity. In this regard, the use of molecular beacons (see Fang, X. H. et al., “Molecular beacons—Novel fluorescent probes,” Analytical Chemistry, 72:747A-753A (2000) and Tyagi, S. & Kramer, F. R., “Molecular beacons: Probes that fluoresce upon hybridization,” Nature Biotechnology, 14:303-308 (1996)) as visible probes has potential for use as a tool to interrogate mRNA in living cells.

However, when used in living cells, molecular beacon hybridization suffers some limitations. First, design of a molecular beacon to target mRNA sequence requires expertise and tedious secondary structure computation. Second, due to nuclease degradation, protein binding (see Li, J. W. J. et al., “Molecular beacons: A novel approach to detect protein—DNA interactions,” Angewandte Chemie-International Edition 39:1049-+(2000) and Tan, W. H. et al., “Molecular beacons: A novel DNA probe for nucleic acid and protein studies,” Chemistry-A European Journal, 6:1107-1111 (2000)) and some thermodynamic fluctuation, molecular beacons tend to generate significant false-positive signals inside cells (see Santangelo, P. J. et al., “Dual FRET molecular beacons for mRNA detection in living cells,” Nucleic Acids Research, 32(b):e57 (2004)). Furthermore, false negative signal exists are often the result of sticky-end pairing (see Li, J. W. J. & Tan, W. H., “A real-time assay for DNA sticky-end pairing using molecular beacons,” Analytical Biochemistry, 312:251-254 (2003)). And finally, most molecular beacons require repeated HPLC purification or PAGE, thus the manufacture of molecular beacons as probes both tedious and expensive.

Accordingly, a molecular probe is needed that can bind to a target nucleic acid sequence, in particular mRNA sequences, in vivo to produce a detectable signal for use in assay methods, diagnostic procedures, and other analytical procedures.

### BRIEF SUMMARY OF THE INVENTION

To meet the demand for sensitive and selective monitoring of mRNA in vivo and to overcome limitations of molecular beacons as sited above, the subject invention provides a new nucleic acid probe, a hybrid molecular probe (sometimes referred to herein as HMP), for detecting nucleic acids. The subject invention utilizes two strands of oligonucleotides to recognize target nucleic acid sequences. The two strands of oligonucleotides are tethered together with a polyethylene glycol polymer. Attached to the ends of the oligonucleotide are fluorophores, which emit a detectable signal upon binding of the probe to a target sequence.

The subject invention further provides methods using an HMP of the invention for analyzing substances, preferably RNA materials, in vivo. For example, a probe of the invention can be used for in vivo detection of nucleic acids without any need of separation. Since the probe is visibly detectable, it can be used during PCR to monitor in real-time the progress of template amplification. It is also useful as a probe for DNA array applications. When used as a probe on an array surface, it allows fast detection of target nucleic acid with excellent sensitivity and specificity and removes the necessity of a tedious washing process.

It is an object of the invention to provide hybrid molecular probes that have the following features: 1) they are easy to design, 2) they allow detection without separation of nucleotide strands, and 3) they afford high sensitivity and good selectivity.

In one embodiment of the invention, an HMP has two single-stranded DNA sequences separated by a polyethylene glycol (PEG) polymer that functions as a linker to tether the two single strands of nucleic acid sequences together. A fluorophore is attached to either end of the probe. In the presence of a target nucleic acid sequence, the probe of the invention hybridizes to the target sequence and brings the two fluorophores within close proximity, allowing fluorescence resonance energy transfer to occur between two fluorophores and enable visible detection by the user. Preferably, the fluorescence changes of these two fluorophores are proportional to target concentrations.

### BRIEF DESCRIPTION OF THE FIGURES

FIG. 1 illustrates a hybrid molecular probe of the invention as it hybridizes to a target sequence.

FIG. 2 is a chart illustrating the effect of the length of the linker molecule on the ability of a probe of the invention to hybridize to its target sequence.

FIG. 3 is a graphical illustration of the correlation between the intensity of a probe's signal emission and the distance between the two strands of nucleotide sequences on the probe.

FIG. 4A is a graphical illustration of the correlation between the fluorescence emission and the presence of a linker molecule.

FIGS. 4B and 4C are illustrations of probes of the invention with or without linker molecules.

FIGS. 5A and 5B are graphical illustrations of the signal enhancement of a probe of the invention and a molecular beacon when in the presence of a target sequence.

FIG. 6 is a graphical illustration comparing the response of a probe with that of a molecular beacon when in the presence of a target sequence.

FIG. 7 is a graphical illustration of the resultant fluorescence emission when a target or control sequence hybridizes with a hybrid molecular probe of the invention.

FIG. 8 is an illustration of a DNA FRET probe of the invention that is immobilized on a solid surface for target sequence detection.

FIG. 9 is a graphical illustration of hybridization of surface immobilized probes of the invention to target sequences.

### BRIEF DESCRIPTION OF THE SEQUENCES

SEQ ID NO. 1 is a nucleic acid sequence of a target for the HMPTBL16 probe of the invention.

SEQ ID NO. 2 is a nucleic acid sequence of a target for a probe of the invention.

SEQ ID NO. 3 is another nucleic acid sequence of a target for a probe of the invention.

SEQ ID NO. 4 is yet another nucleic acid sequence of a target for a probe of the invention.

SEQ ID NO. 5 is another nucleic acid sequence of a target for a probe of the invention.

SEQ ID NO. 6 is a nucleic acid sequence that was synthesized to target Tublin mRNA(516-551.

SEQ ID NO. 7 is a nucleic acid sequence of a probe (HMPTBL16 probe) of the invention.

SEQ ID NO. 8 is a nucleic acid sequence of a control for the HMPTBL16 probe of the invention.

### DETAILED DESCRIPTION OF THE INVENTION

The present invention provides novel hybrid molecular probes that specifically bind to a target nucleic acid sequence to produce a detectable signal as well as methods for their production and use. The hybrid molecular probes of the invention comprise at least two oligonucleotide strands, a linker polyethylene glycol (PEG) polymer, and at least two fluorophores, wherein the fluorophores can be linked at any position within the hybrid molecular probes provided that the point of attachment of fluorophores are located at opposite ends of the oligonucleotide strands. The PEG polymer serves to link the two oligonucleotide strands to each other.

The fluorophores are preferably attached to the ends or near the ends of each oligonucleotide strand. When a probe of the invention hybridizes with a target nucleic acid sequence, the probe undergoes a conformational change to bring the fluorophores closer in proximity to each other. This change in distance causes a change in the photon absorption or emission of the fluorophores, creating a visual indication that the probe of the invention has bound a target sequence.

The oligonucleotide strands of the probe can encompass single and double-stranded RNA, single and double-stranded DNA and cDNA, nucleic acid analogs, aptamers, and the like. The term nucleic acid and oligonucleotide are used interchangeably herein. Preferably, the oligonucleotide strands of the probe are single-stranded DNA.

The PEG linker of the invention can be any suitable PEG polymer that provides a flexible polymeric backbone to allow free movement of the oligonucleotide strands without interfering with the ability of the oligonucleotide strands to bind to a target sequence. According to the subject invention, the number of PEG monomer units used will be determined empirically and may vary from probe to probe, and with the target sequence.

In certain embodiments, polypeptides can be inserted into the PEG linker of the invention to optimize the distance between the oligonucleotide strands. In one embodiment, at least one biotin is inserted into the PEG linker of the invention for surface immobilization purposes.

In a preferred embodiment, two single-stranded DNA sequences are tethered together by PEG and have a fluorophore attached at the ends of the DNA sequences. Fluorescent resonance energy transfer (FRET) or non-FRET interactions are used to detect the binding of the target sequence to the ssDNA sequences of the invention. FRET interactions (also known as non-radiative energy transfer; see Yaron et al., Analytical Biochemistry 95:228-235 (1979)) for quenching fluorescence signals requires spectral overlap between the donor and acceptor fluorophore moieties and the efficiency of quenching is directly proportional to the distance between the donor and acceptor moieties of the FRET pair. Extensive reviews of the FRET phenomenon are described in Clegg, R. M., Methods Enzymol, 221: 353-388 (1992) and Selvin, P. R., Methods Enzymol, 246: 300-334 (1995). In contrast, non-FRET interactions (also known as radiationless energy transfer; See: Yaron et al., Analytical Biochemistry 95:228-235 (1979)) requires short range interaction by “collision” or “contact” between the fluorophore moieties and therefore requires no spectral overlap between the donor and acceptor pair.

When the probe binds to the target sequence, the probe will undergo a conformational change causing the distance and/or angle between the fluorophore pairs to change. This change can then be detected because it will change the efficiency of resonance energy transfer between the fluorophore moieties after exposure of the probe to an excitation wave-length of light.

### Design of Hybrid Molecular Probe

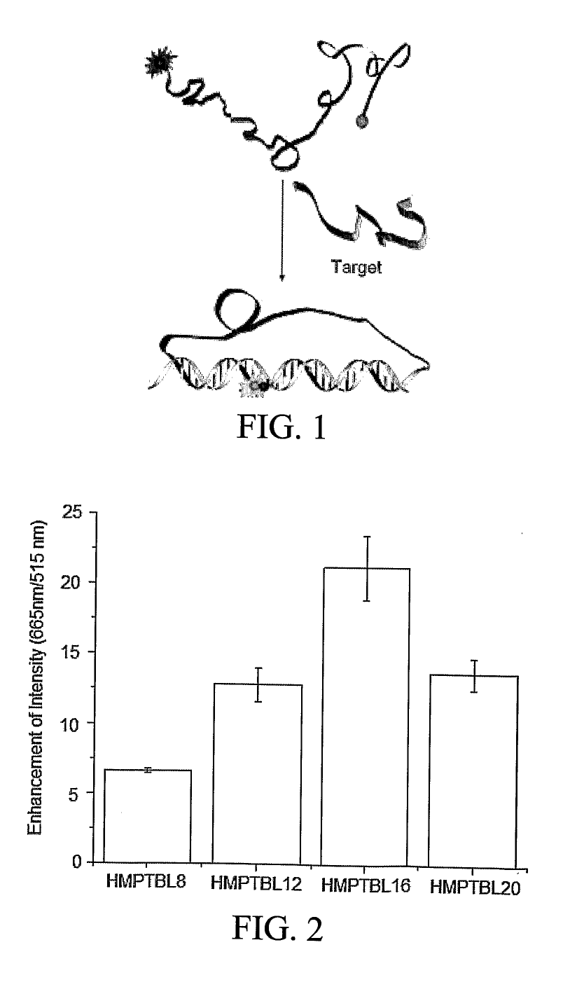

In one embodiment, as illustrated in FIG. 1, a probe of the invention consists of two single-stranded DNA sequences tethered to a PEG polymer linker having a controlled length. The probe consists of two single-stranded DNA, illustrated as green ribbons, which are tethered to either ends of a PEG polymer linker with a controlled length, illustrated as a purple ribbon. A donor fluorophore, illustrated as a green ball, is attached to the 3′ end and an acceptor fluorophore to the 5′ end of the probe DNA. Due to the random coil structure of these two DNA strands in the probe, the two fluorophores are separated from each other in the absence of a target nucleotide sequence. Thus, when the donor fluorophore is excited, only the donor fluorophore gives a fluorescence emission, while the acceptor has no emission because of the distance separating the donor and acceptor fluorophores. In the presence of a target, the probe of the invention hybridizes to the target and brings the two fluorophores into close proximity, which allows fluorescence resonance energy transfer (FRET) (see Lakowicz, J. R., Principles of Fluorescent Spectroscopy, Plenum US (1986)) to occur. Hence, target hybridization results in the quenching of the donor fluorophore and enhancement of the acceptor fluorescence.

### Optimization of PEG Linker Length

The role of PEG in a probe of the invention is its ability to tether two DNA sequences together to increase their local concentrations to each other and to help the two sequences bind to the same target sequence instead of different sequences. The length of this linker group should be carefully optimized to allow these two sequences to freely bind to their target, while still allowing the two sequences to have relatively high local concentration to each other.

Four probes, HMPTBL8, HMPTBL12, HMPTBL16 and HMPTBL20, were prepared. The number in the probe name indicates the number of PEG monomer units in the probe.

As shown in FIG. 2, when only 8 repeat PEG units were used, as high as a 7 fold signal enhancement was observed. An increase of signal enhancement was observed when the PEG spacer length increased.

For probes with short linkers, there are two factors that make short linkers unfavorable for good signal enhancement. First, FRET could occur between acceptor and donor, which would contribute to background signal. For instance, for HMPTBL8, it had 30 bases and 8 PEG units, the distance between the two fluorophores could be as high as 27.6 nm if the probe was fully stretched. However, the distance was shorter as free probe tended to be in coiled form.

Secondly, when the linker length was short, it hindered the ability of the two DNA sequence strands to hybridize to its target, weakening probe-target hybrid stability. However, this doesn't mean the longer the linker, the better. When the linker is too long, such as with the KLP20 probe with 20 PEG units, it will decrease the local concentration of one DNA to the other on the same probe, lowering the chance of the two DNA sequences to binds to the same target sequences. As a consequence, HMPTBL20 had a slightly lower signal enhancement than HMPTBL16.

### Optimization of Acceptor-Donor Distance in Target-Bound Probe

The optimization of the Acceptor-Donor distance was carried out with a goal to minimize static quenching, while maximizing FRET efficiency. Binding of the probe to its target, as designed, will bring acceptor and donor fluorophore within close proximity to allow FRET to occur. In the case of FRET, the closer the two fluorophores are, the higher the energy transferring efficiency.

On the other hand, however, if two fluorophores get too close to each other, static quenching will happen, which will quench both fluorophores. To get a best signal enhancement, it is important to optimize the distance between these two fluorophores. Different numbers of dTs were inserted into a DNA sequence that was complementary to the linear probe so that the distance of the acceptor and donor varied once the probe bound to these DNAs. Table 1 shows the sequences that were used in this experiment. For FAM/CY5 pair, there was not static quenching observed. Instead, the FRET efficiency decreased exponentially with increase of FAM/CY5 distance in target/probe hybrid.

TABLE 1
Target DNA sequence with different numbers of dT

| | | |
|---|---|---|
| 0T Target | GCT CAT GAG CAA AAT GAG GGAGGAGTACCCAGACAG | SEQ ID NO. 1 |
| 1T Target | GCT CAT GAG CAA AAT GAG T GGAGGAGTACCCAGACAG | SEQ ID NO. 2 |
| 3T Target | GCT CAT GAG CAA AAT GAG TTT GGAGGAGTACCCAGACAG | SEQ ID NO. 3 |
| 5T Target | GCT CAT GAG CAA AAT GAG TTT TT GGAGGAGTACCCAGACAG | SEQ ID NO. 4 |
| 7T Target | GCT CAT GAG CAA AAT GAG TTTTTTTGGAGGAGTACCCAGAGAG | SEQ ID NO. 5 |

### Calibration Curve of a Hybrid Molecular Probe

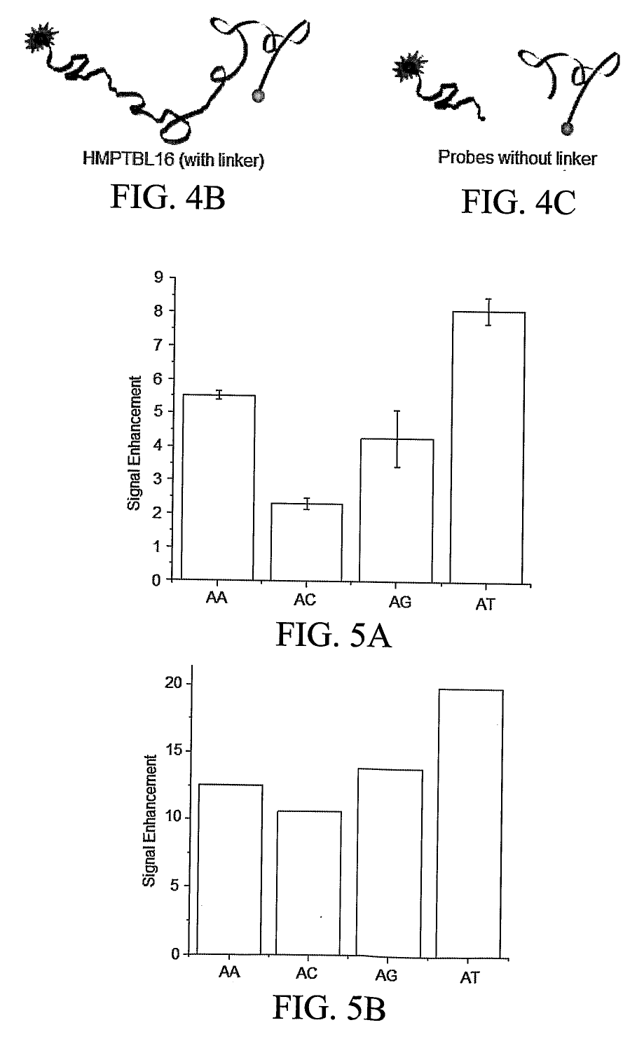

Probe HMPTBL16 was titrated with different concentrations of target DNA to check the probe's sensitivity and its dynamic range. Signal response from HMPTBL16 was proportional to target DNA concentration ranging from 0 to 500 nM. As a comparison, two DNA probes without a linker group were titrated with the same target DNA. For two probes without linker (see FIG. 4C), the fluorescence intensity ratio 665 nm/515 nm the ratio decreased after target to probe ratio was larger that 1 to 1 ratio. This nonlinear response shows the disadvantage of this two probe system. This nonlinear response at high target concentration comes from the fact that the two probes tend to bind to two separated target DNA when the target is in excess.

In contrast, the linear FRET probe uses linker to tether two individual sequences together (see FIG. 4B), increasing local concentration of each other, thus ensuring that two individual DNA sequences in the same probe bind to a same target DNA sequence. As a consequence, even at higher target to probe concentration ratio, the signal response is still close to linear (see FIG. 4A). Dual FRET molecular beacon approach has been developed to reduce false positive signal for molecular beacon in mRNA imaging in living cells (Philip J Santangelo, and Gang Bao et al, Nucleic Acid Research, 2004, 12, e57). However, one of major problem of the dual FRET beacon approach is that the chance of getting two beacons to bind to two adjacent areas is low and the likelihood of nonlinear signal response at high target concentration.

A HMP of the invention that uses a PEG linker to tether two molecular beacons can effectively be used to solve the aforementioned problems. Another advantage of HMP over dual linear FRET probes is that without a linker, tighter binding of probe to target molecules is enabled because of an increase in local concentration effect. Indeed, the melting temperature of a HMP-cDNA hybrid of the invention was found to be more than 20° C. higher than that of a hybrid made of target with two separate probes without a linker.

FIG. 4A is a graphical illustration of the results from titration of 300 nM HMPTBL16 and 300 probes without a linker with target DNA in 20 mM Tris-HCl buffer (50 mM NaCl, 5 mM MgCl₂, pH 7.5). Excitation occurred at 488 nM in temperatures of 25° C.

### HMP for Surface DNA Hybridization Study

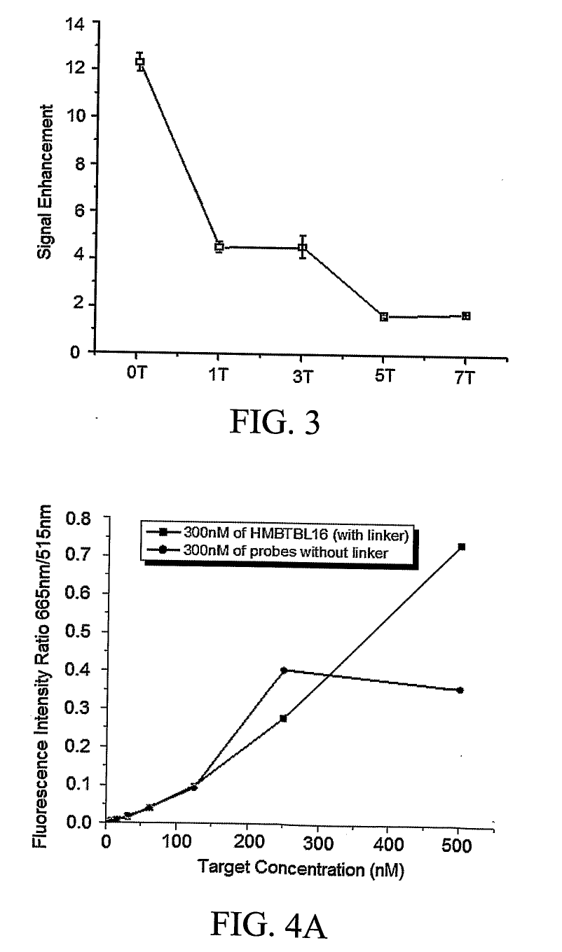

One of the advantages of the subject probe over conventional two-probe FRET system or Dual FRET MBs design is a larger dynamic range. Another advantage of using the linear probe of the invention is that it can be used for surface hybridization applications such as fiber optic DNA sensor, DNA array, as well as microchannel for nucleic acid detection (FIG. 3).

Comparison of HMP with Molecular Beacons

Molecular Beacons have excellent selectivity, especially for single base mismatch discrimination capability. A molecular beacon targeting the same target sequence as that of HMPTBL16 was prepared and used in single based mismatch detection experiments. FIGS. 5A and 5B show the response of 300 nM of a molecular beacon to 300 nM of different targets. Under experimental conditions, perfect match cDNA was able to produce a signal change of about 8.5 folds. When the molecular beacon was exposed to one base mismatch target present in the same concentration, the molecular beacon produced less of a signal change. For example, when the nucleic acid T was changed to G, the target DNA emitted a change in signal was about 4.5 fold in difference. With A replacing T in the target sequence, target DNA produced about a 5.5 times difference in signal emission. Target DNA change in sequence where C replaced T produced only about 2.5 times difference in signal emission.

In the case of the probe of the invention, it generated about a 20 times change in signal upon hybridization to a perfectly matched target DNA. While one base mismatch, such as AT, AC, and AG targets, was able to lead to about 12.5, 11, and 14 times difference in signal emission, respectively. Overall, under the same experimental conditions, the signal enhancement of a hybrid molecular probe of the invention is greater than that of a molecular beacon. According to the subject invention, the selectivity of a molecular probe can be modified by designing a probe with DNA sequences that adapt a hair-pin structure.

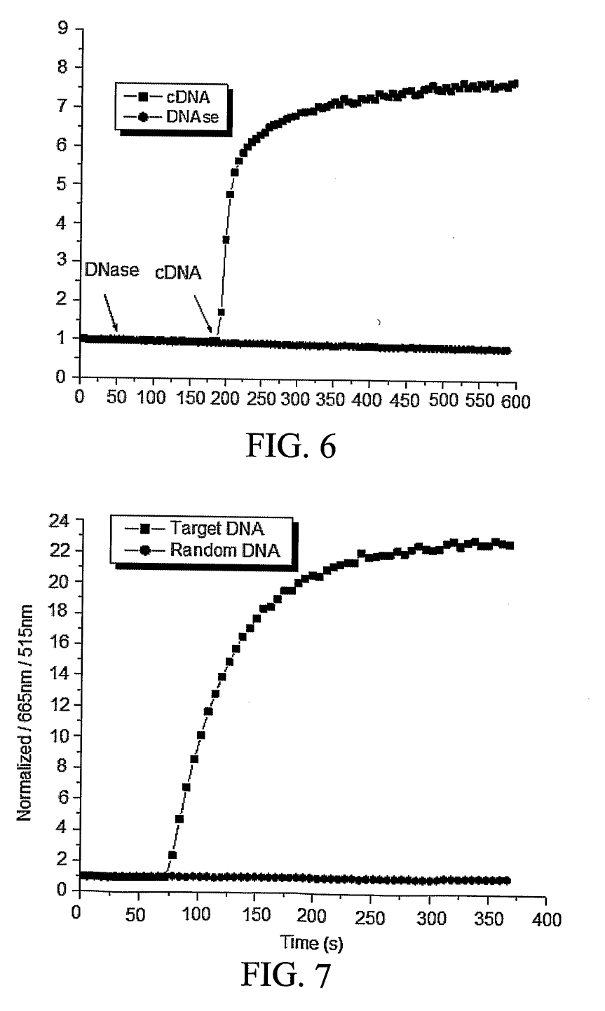

For a molecular beacon, one of its disadvantages is that it gives a false-positive signal after exposure to protein binding, enzyme digestion, or thermal denaturing. In contrast, a hybrid molecular probe of the invention does not emit any positive signals after exposure to any of these factors. This is emphasized in FIG. 6, which compares the response of a molecular beacon and an HMP of the invention to a target DNA in the presence of nucleases. For the HMP, no false positive signals were observed when the nuclease was added to the solution. In contrast, digestion of the molecular beacon by a nuclease caused intense false positive signals that were indistinguishable from true target binding signals.

The probe of the subject invention is unique in that it responds specifically to its complementary sequence. It allows a rapid detection of an unlabeled target sequence without separation. This property offers convenience for many applications, especially for in vivo real-time detections where separation is impossible. In solution, this probe responds to its target sequences with high signal enhancement. Using avidin-biotin interaction, this probe was immobilized on solid surface to assess probe selectivity to a target sequence.

The probe of the invention is particularly advantageous in that: 1) they are visually detectable; 2) they can detect unlabeled target sequences without separation; 3) the detectable emission, upon binding with a target sequence, is high; and (4) the probes demonstrate high affinity and selectivity.

Compared to molecular beacon, however, this subject probe has its own advantages. First, it is far easier to design. Second, it generates a higher signal-to-noise ratio when immobilized on a surface. Third, the signal transduction is based on ratiometric measurement, which minimizes the effect of the system fluctuations. And finally, the probe of the invention will not generate any false-positive signals upon digestion by nuclease. Accordingly, by constructing DNA/mRNA sensors in nanometer size using an HMP, the subject invention is useful in monitoring expression, distribution, and trafficking of mRNA in single living cells (see Kopelman, R. & Tan, W. H., “Near-Field Optics—Imaging Single Molecules,” Science 262, 1382-1384 (1993) and Tan, W. H., et al., “Submicrometer Intracellular Chemical Optical Fiber Sensors,” Science 258, 778-781 (1992)). Further, the probe of the invention is applicable to microjection/microscopy imaging for mRNA monitoring.

In yet another embodiment, this invention is directed to kits suitable for performing an assay which detects the presence, absence or amount of one or more target sequence which may be present in a sample. The characteristics of a probe suitable for the detection, identification or quantitation of amount of one or more target sequences have been previously described herein. The kits of the subject invention comprise one or more hybrid molecular probes and other reagents or compositions which are selected to perform an assay or otherwise simplify the performance of an assay. Preferred kits contain sets of hybrid molecular probes, wherein each of at least two hybrid molecular probes of the set are used to distinctly detect and distinguish between the two or more different target sequences which may be present in the sample. Thus, the hybrid molecular probes of the set are preferably labeled with independently detectable fluorophore moieties so that each of the two or more different target sequences can be individually detected, identified or quantitated (a multiplex assay).

Following are examples that illustrate procedures for practicing the invention. These examples should not be construed as limiting. All percentages are by weight and all solvent mixture proportions are by volume unless otherwise noted.

### EXAMPLE 1

### Hybrid Molecular Probe

To demonstrate how a hybrid molecular probe of the invention functions, the following sequence was synthesized to target Tublin mRNA(516-551): 5′-Cy5-CTC ATT TTG CTG ATG AGC-(X)ₙ-CTG TCT GGG TAC TCC TCC-FAM-3′ (SEQ ID NO. 6), where X stands for a PEG synthesizing monomer unit (Glen Research, Sterling, Va.), n represents the number of PEG monomer units. Every PEG monomer unit has a length of about 22 Å and the PEG linker is flexible so as to allow free movement of the two DNA sequences.

Several criteria are considered when selecting a donor/acceptor fluorophore pair for a probe of the invention to ensure good signal-to-background ratio. First, there should be a significant spectra overlap between the donor and acceptor dyes. Second, absorption of acceptor at donor excitation should be negligible. And finally, their fluorescence emission spectra should be completely separated and hence any false positive signal from the acceptor is considerably reduced at the donor excitation wavelength.

Several dye pairs, such as FAM/CY5, BODIPY FL/CY5, BODIPY FL/TEXAS RED, and CY3/CY5 meet these criteria. In this Example, FAM and Cy5 were chosen. The probe was synthesized on an ABI3400 DNA/RNA synthesizer (Applied Biosystems, Foster City, Calif.). A ProStar HPLC (Varian, Walnut Creek, Calif.) was used for probe purifications using C18 column (5 um, 60 Å, 4.6×250 mm, Deerfield, Ill., Alltech).

The response of 300 nM HMPTBL16 (a hybrid molecular probe of the subject invention that targets Tublin mRNA with 16-unit spacer) to 300 nM of its target DNA is illustrated in FIG. 2. Hybridization of 300 nM of HMPTBL16 to 300 nM of its target DNA and Control was performed in 20 mM Tris-HCl buffer (50 mM NaCl, 5 mM MgCl₂, pH 7.5).

The HMPTBL16 probe sequence is as follows: Cy5-CTC ATT TTG CTG ATG AGC (X)₁₆CTG TCT GGG TAC TCC TCC-FAM (SEQ ID NO. 7). The target sequence is as follows: GCT CAT CAG CAA AAT GAG GGA GGA GTA CCC AGA CAG (SEQ ID NO. 1). The control scrambled sequence is as follows: TCT GTG TAA TCA ACT GGG AGA ATG TAA CTG ACT AGC (SEQ ID NO. 8). As shown in FIG. 2, excitation occurred at 488 nM, with the temperature at 25° C.

When excited at 488 nm, the probe of the invention emitted strongly with a 515 nm peak in the absence of target DNA, while the Cy5 emission at 655 nm was negligible. When a target DNA was added to the solution, the probes of the invention hybridized to the target, causing the two fluorophores to be brought within close proximity to each other and enabling FRET to occur. As a consequence, fluorescence of FAM decreased and emission of Cy5 increased. This is an indication that this probe works as expected.

To confirm the response was due to specific DNA hybridization, a scrambled DNA sequence was used as a control target in the hybridization experiment. No significant signal change was observed from the HMBTBL20 when the same concentration of a scramble (control) sequence was added, which suggested the positive response from c-DNA was the result of DNA/DNA hybridization.

Data shown in FIG. 7 reveals two important advantages of the probes of the invention: (1) detection-without-separation and (2) ratiometric measurement. The fluorescence intensity ratio of CY5/FAM from free probe is very low. By contrast, when the probe of the invention hybridizes to its target, the ratio of CY5/FAM is high. This ‘light-up’ signaling approach allows the probe of the invention to detect the presence of target sequence without any need for removing unbound probes. This detection-without-separation method eliminates tedious washing and separating procedures. More importantly, it affords real-time detection, which is desirable as a probe for in vivo detection in living cells.

Another advantage of the probe of the invention is its use for ratiometric measurement. By taking the intensity ratio of the CY5 emission to FAM emission, one could effectively eliminate signal fluctuation and minimize impact of environmental quenching on the accuracy of measurement.

### EXAMPLE 2

### Hybrid Molecular Probe Immobilization

A HMP probe was prepared with the same sequence as HMPTBL except that there were two biotins inserted in the middle of linker PEG units. Two biotins were used for one sequence to improve the binding efficiency. Before immobilization onto a streptavidin-coated surface, a solution test was performed, which showed similar signal response of the probe to the probe without biotin. This indicated that the inserted biotin in between the linker did not interfere the binding of probe to its target.

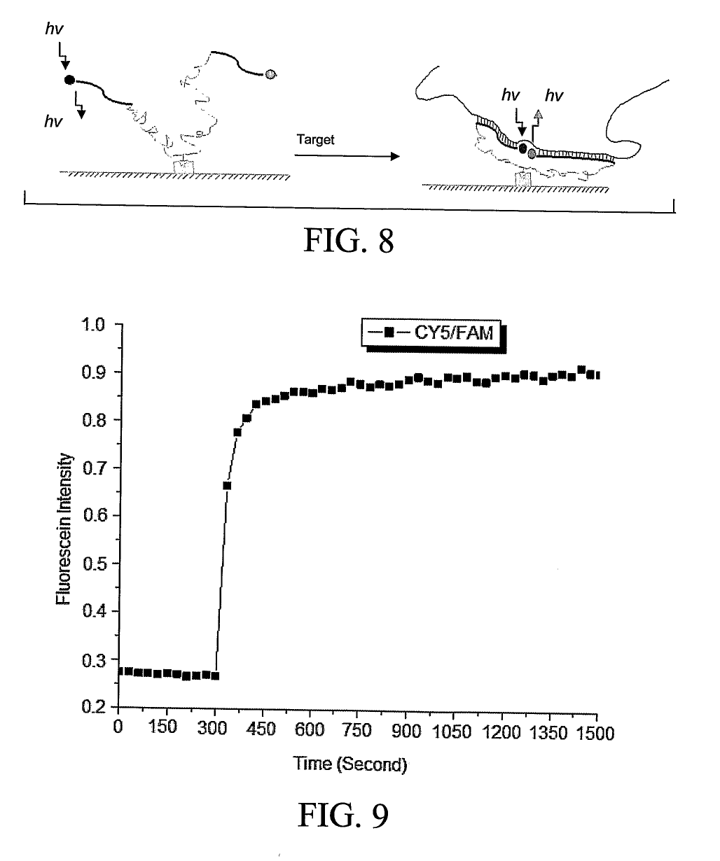

FIG. 8 is the response of the immobilized probe upon the addition of target DNA. The surface was excited at 488 nm, and the images were monitored at two emission channels specific for FAM and Cy5 respectively. Before the hybridization, fluorescence signal from FAM was strong and week emission from Cy5 was observed. Immediately after addition of target c-DNA, the intensity of FAM diminished and the intensity of Cy5 increased as a result of hybridization. Overall, fluorescent intensity ratio of Cy5/FAM increased dramatically (see FIG. 9). From the intensity results, large fluctuation for both Cy5 and FAM was observed. This fluctuation was a result of disturbance of the detection system. By taking ratiometric, this noise was cancelled out, and smooth hybridization results were observed. With ratiometric measurement capability, the new DNA probe design removes the internal fluctuations of the detection system, allowing a more precise detection.

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A probe having a high specifically for a target nucleic acid sequence, wherein the probe comprises at least two oligonucleotide strands, a polyethylene glycol (PEG) polymer, and at least two fluorophores.

2. The probe of claim 1, wherein the PEG links the two oligonucleotide strands together.

3. The probe of claim 2, wherein the at least two oligonucleotide strands comprise a first strand and a second strand, wherein the at least two fluorophores comprises a first fluorophore and a second fluorophore, wherein the first fluorophore is attached to the first strand, and the second fluorophore is attached the to the second strand.

4. The probe of claim 2, wherein the at least two oligonucleotide strands comprise a first strand and a second strand, wherein the at least two fluorophores comprises a first fluorophore and a second fluorophore, wherein the first fluorophore is attached to the end of the first strand, and the second fluorophore is attached to the end of the second strand.

5. The probe of claim 2, wherein the at least two fluorophores are selected from the group consisting of: FAM, CY5, CY3, BODIPY FL, TEXAS RED, and any combinations thereof.

6. The probe of claim 2, wherein the probe is selected from the group consisting of: single stranded RNA, double stranded RNA, single stranded DNA, double stranded DNA, nucleic acid analogs, and any combinations thereof.

7. The probe of claim 2, wherein the PEG polymer provides a flexible polymeric backbone to allow free movement of the oligonucleotide strands without interfering with the ability of the oligonucleotide strands to bind to said target sequence.

8. The probe of claim 7, wherein at least one polypeptide is inserted into the PEG polymer to optimize the distance between the oligonucleotide strands.

9. The probe of claim 8, wherein the polypeptide is at least one biotin.

10. A method for detecting a target nucleic acid sequence comprising:
a) applying to a sample comprising nucleic acid sequences a probe comprising at least two oligonucleotide strands, a polyethylene glycol (PEG) polymer, and at least two fluorophores, wherein the at least two fluorophores each emit a fluorescence emission and wherein fluorescence resonance energy transfer (FRET) occurs upon binding of the oligonucleotide strands to the target nucleic acid sequence; and
b) detecting FRET, which would indicate the presence of the target nucleic acid sequence.

11. The method of claim 10, wherein the step of detecting FRET is accomplished using a sensor selected from the group consisting of: fiber optic DNA sensor, DNA array, microchannel for nucleic acid detection, and any combination thereof.

12. The method of claim 10, wherein the PEG links the two oligonucleotide strands together.

13. The method of claim 12, wherein the at least two oligonucleotide strands comprise a first strand and a second strand, wherein the at least two fluorophores comprises a first fluorophore and a second fluorophore, wherein the first fluorophore is attached to the first strand, and the second fluorophore is attached to the second strand.

14. The method of claim 12, wherein the at least two oligonucleotide strands comprise a first strand and a second strand, wherein the at least two fluorophores comprises a first fluorophore and a second fluorophore, wherein the first fluorophore is attached to the end of the first strand, and the second fluorophore is attached to the end of the second strand.

15. The method of claim 12, wherein the at least two fluorophores are selected from the group consisting of: FAM, CY5, CY3, BODIPY FL, TEXAS RED, and any combinations thereof.

16. The method of claim 12, wherein the probe is selected from the group consisting of: single stranded RNA, double stranded RNA, single stranded DNA, double stranded DNA, nucleic acid analogs, and any combinations thereof.

17. The method of claim 12, wherein the PEG polymer provides a flexible polymeric backbone to allow free movement of the oligonucleotide strands without interfering with the ability of the oligonucleotide strands to bind to said target sequence.

18. The method of claim 17, wherein at least one polypeptide is inserted into the PEG polymer to optimize the distance between the oligonucleotide strands.

19. The method of claim 18, wherein the polypeptide is at least one biotin.
